(19) United States
(12) Patent Application Publication
(10) Pub. No.: US 2023/0298719 A1
(43) Pub. Date: Sep. 21, 2023

(54) A System and Method for Developing an Alternative Drug Therapy using Characteristics of an Existing Drug Therapy to Produce a Similar Pathway Behavior

(71) Applicant: Inderpreet Jalli, Houston, TX (US)

(72) Inventor: Inderpreet Jalli, Houston, TX (US)

(21) Appl. No.: 18/017,076

(22) PCT Filed: Jul. 19, 2021
(86) PCT No.: PCT/US21/42263

§ 371 Date: Jan. 19, 2023

Related U.S. Application Data
(60) Provisional application No. 63/053,706, filed on Jul. 19, 2020.

(51) Int. Cl. G16H 20/10; G16B 5/00; G16B 15/30; G16H 50/50

(52) U.S. Cl. CPC G16H 20/10; G16B 5/00; G16B 15/30; G16H 50/50

(57) ABSTRACT

A method for developing a new drug therapy using characteristics of an existing drug therapy, the method comprising the following steps: developing a new-treatment mathematical model of a targeted biological network and synthesizing a drug therapy based on the new-treatment mathematical model. The new treatment mathematical model can be capable of producing a new-treatment time-course progression comprising a time-course progression signature found in an existing-treatment time-course progression of an existing treatment mathematical model of the targeted biological network. The time-course progression signature can be related to an outcome of said targeted biological network. A drug regimen can be synthesized for each the new treatment intervention constant, and the drug regimens together can be a new drug therapy.

### BACKGROUND

This disclosure relates to a system and method for developing an alternative drug therapy using characteristics of an existing drug therapy to produce a similar pathway behavior.

Today, drug therapies are used to treat pathogens and diseases. Drug therapies work by attacking along particular pathways of the pathogen. Generally speaking, a pathway is a causal chain of interactions that lead to the alteration of the normal functioning of a pathogen set off by a drug therapy chemically interacting with targetable biological elements of the pathogen.

While many drug therapies exist, much further exploration is being performed to find new drug therapies, either to fight pathogens for which no drug therapies yet exist or to replace current inadequate drug therapies. Drug therapies can be inadequate for a number of reasons.

First, some drug therapies do not cure a disease, but merely reduce prevalence or symptoms. Examples of such drug therapies ones used to fight HIV or herpes viruses. In both cases, while drug therapies reduce viral loads within a person, none of the drug therapies completely eliminate the virus.

Second, some drug therapies have side effects ranging from mild to severe, and in some cases, life-threatening. The biological elements targeted by drug therapies results to an altered pathway behavior of the targeted elements, and consequently, its interaction with the other biological elements as a result of a chain reaction. These altered pathway behaviors, however, can produce significant negative effects to the biological network. Moreover, the therapeutic molecule can interact with either known or unknown non-target elements in the network which can also produce a negative overall pathway behavior in the target network as above described.

Third, drug therapies are often prone to being resisted by evolving pathogens over time. Specifically, in the case of therapeutic agents that target pathogenic organisms such as bacteria, viruses, parasites, or even cancer cells, therapeutic agents can lose efficacy due to evolution in target populations. Resistance occurs when a subset of a targeted set of organisms or cells survive exposure due to a particular trait of that subset, and then pass on such resistant trait to a next generation.

Fourth, some drug therapies can be expensive to make. Drug synthesis is a multistep process, and one step can have considerable effects on the cost to manufacture a drug. For example, in 2011, 4-Phenyl-1, cost $260 to make just 50 grams.

A common strategy for rational computer-aided development of new drugs is to first identify new interactions between biological elements or entirely new biological elements that may be critical to cell function. Highly promising biological elements are then structurally characterized at the molecular level along with their interaction with potential therapeutic agents. The aim is to target a specific biological element that has a high probability of significantly altering the function of the target cell in the desired manner.

However, such methods pose significant problems. Discovery of new biological elements or interactions within known elements is extremely time consuming and resource-intensive, or filled with false positives for interaction results. A discovery of an interaction or biological element that is critical to cell function in initial lab tests is also unable to answer a central question: does disruption of the target biological element lead to the desired effect on the organism overall via the chain reaction mechanism?

As such, it would be advantageous to have a system and method for designing a novel drug therapy targeting a pathway of a pathogen using characteristics of an existing drug therapy to produce similar pathway behavior.

### SUMMARY

A method for finding a set of parameters for a new drug therapy such that the new drug therapy produces an outcome similar to an existing drug therapy. In a first step, the method can comprise replacing, within a mathematical model any intervention functions related to an existing drug therapy with untreated-node functions related to the mathematical model, if the mathematical model has any such intervention functions. In a next step, the method can comprise choosing parameter ranges for each of a plurality of parameters. Next, the method can comprise, producing a time course progression for a new-treatment mathematical model for each permutation of a plurality of permutations, each time, using the permutation to produce the time course progression. Next, the method can comprise determining for each permutation whether its time-course progression comprises a time-course progression signature present in an existing-treatment time-course progression related to the existing drug therapy, the time-course progression signature related to an outcome of the existing drug therapy. Next, the method can comprise, for at least one permutation comprising the time-course progression signature, synthesizing substances having kinetic properties substantially matching kinetic parameters of that one permutation, to produce a new drug therapy.

A method for developing a new drug therapy using characteristics of an existing drug therapy, the method comprising the following steps: developing a new-treatment mathematical model of a targeted biological network and synthesizing a drug therapy based on the new-treatment mathematical model. The new treatment mathematical model can be capable of producing a new-treatment time-course progression comprising a time-course progression signature found in an existing-treatment time-course progression of an existing treatment mathematical model of the targeted biological network. The time-course progression signature can be related to an outcome of the targeted biological network. The new-treatment mathematical model can comprise a new-treatment intervention function modeling each new-treatment intervened-upon node of a set of new-treatment intervened-upon nodes, and a first set of no-treatment node-velocity functions modeling all other nodes of the new-treatment mathematical model. Each new-treatment intervention function can comprise one more new-treatment intervention constants from a set of new-treatment intervention constants. The existing-treatment mathematical model can comprise an existing-treatment intervention equation modeling each existing-treatment intervened-upon node of a set of existing-treatment intervened-upon nodes, and a no-treatment velocity equation modeling all other nodes of the existing-treatment mathematical model. Each existing-treatment intervention equation can comprise an existing-treatment intervention constant from a set of existing-treatment intervention constants. The set of new-treatment nodes can be not identical to the set of existing-treatment nodes. Further, the set of new-treatment nodes can comprise at least one node not in the set of existing-treatment nodes. Further, the set of existing-treatment nodes can comprise at least one node not in the set of new-treatment nodes. A drug regimen can be synthesized for each the new treatment intervention constant, and the drug regimens together can be a new drug therapy.

### BRIEF DESCRIPTION OF THE DRAWINGS

FIG. 1 illustrates a drug therapy interacting with a biological network.

FIG. 2 illustrates an exemplary reaction model of biological network.

FIG. 3 illustrates a time-course progression of a biological network, specifically an actual time-course progression.

FIG. 4 illustrates a mathematical model a of biological network, in particular a pre-treatment mathematical model.

FIG. 5 illustrates a pre-treatment time-course progression.

FIG. 6 illustrates an existing-treatment reaction model.

FIG. 7 illustrates an existing-treatment reaction model.

FIG. 8 illustrates an existing-treatment mathematical model.

FIG. 9 illustrates a first set of existing-treatment intervention functions.

FIG. 10 illustrates a set of existing-treatment intervention constants.

FIG. 11 illustrates a set of existing-treatment intervention concentrations.

FIG. 12 illustrates untreated node velocities.

FIG. 13 illustrates an existing-treatment time-course progression.

FIG. 14 illustrates a new-treatment reaction model.

FIG. 15 illustrates new-treatment mathematical model.

FIG. 16 illustrates a second set of new-treatment intervention functions.

FIG. 17 illustrates a set of new-treatment intervention constants.

FIG. 18 illustrates a set of new-treatment intervention concentrations.

FIG. 19 illustrates a second set of untreated node velocity functions.

FIG. 20 illustrates a new-treatment time-course progression.

### DETAILED DESCRIPTION

Described herein is a system and method for developing an alternative drug therapy using characteristics of an existing drug therapy to produce a similar pathway behavior. The following description is presented to enable any person skilled in the art to make and use the invention as claimed and is provided in the context of the particular examples discussed below, variations of which will be readily apparent to those skilled in the art. In the interest of clarity, not all features of an actual implementation are described in this specification. It will be appreciated that in the development of any such actual implementation (as in any development project), design decisions must be made to achieve the designers' specific goals (e.g., compliance with system- and business-related constraints), and that these goals will vary from one implementation to another. It will also be appreciated that such development effort might be complex and time-consuming but would nevertheless be a routine undertaking for those of ordinary skill in the field of the appropriate art having the benefit of this disclosure. Accordingly, the claims appended hereto are not intended to be limited by the disclosed embodiments but are to be accorded their widest scope consistent with the principles and features disclosed herein.

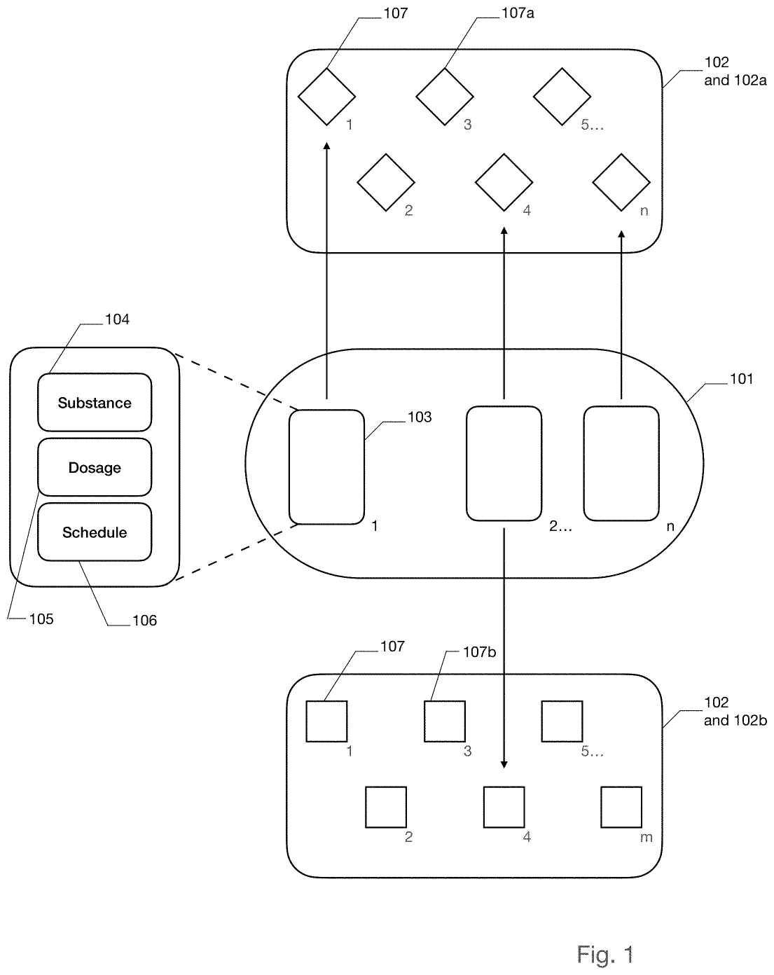

FIG. 1 illustrates a drug therapy 101 interacting with a biological network 102. Within the context of this disclosure, biological network 102 can be a target biological network (TBN) 102a or a non-targeted biological network (non-TBN) 102b. TBN 102a can include, but is not limited to, all or a portion of a pathogen or a disease. TBN 102a can be multicellular, single cellular, or even RNA or DNA. Example categories of TBN 102a can include parasitic animals, bacteria, viruses, or fungi. Specific examples can in include E-coli, COVID-19, or cancer. Non-TBN 102b, for purposes of this disclosure, is biological network 102 within a host or organism in a mutualistic relationship with the host.

This disclosure describes systems and method for developing one or more drug therapies 101 that disrupt TBN 101a. Drug therapy 101 is any one or more drug regimens 103, other than food, that is/are used to prevent, diagnose, treat, or relieve symptoms of a disease or abnormal condition. Further, for purposes of this disclosure, drug regimen 103 can be defined by substance 104. Substance 104, for purposes of this disclosure is a particular kind of matter with uniform properties. Additionally, drug regimen 103 can be defined by dosage 105, and schedule 106. Schedule 106, in one embodiment, can be defined by a period and/or duration (eg., every 8 hours for 3 days). In some embodiments of drug regimen 103, dosage 105 can vary with schedule 106, such as increasing or decreasing over time. For purposes of this disclosure, dosage 105 can be described in absolute amounts, amounts meant to be scaled by other patient-specific information such as weight, age, maturity, etc., as intended concentrations, or as any other method to describe dosage known in the art.

Biological networks 102 comprise nodes 107. For purposes of this disclosure, nodes 107 are aspects of biological networks 102 upon which drug therapy 101 could potentially intervene-upon such as by accelerating, decelerating, preventing, or initiating a chemical interconversion, within biological network 102. Further, for purposes of this disclosure, nodes 107 of TBN 102a are target nodes 107a, and nodes 107 of non-TBN 102b are non-target nodes 107b.

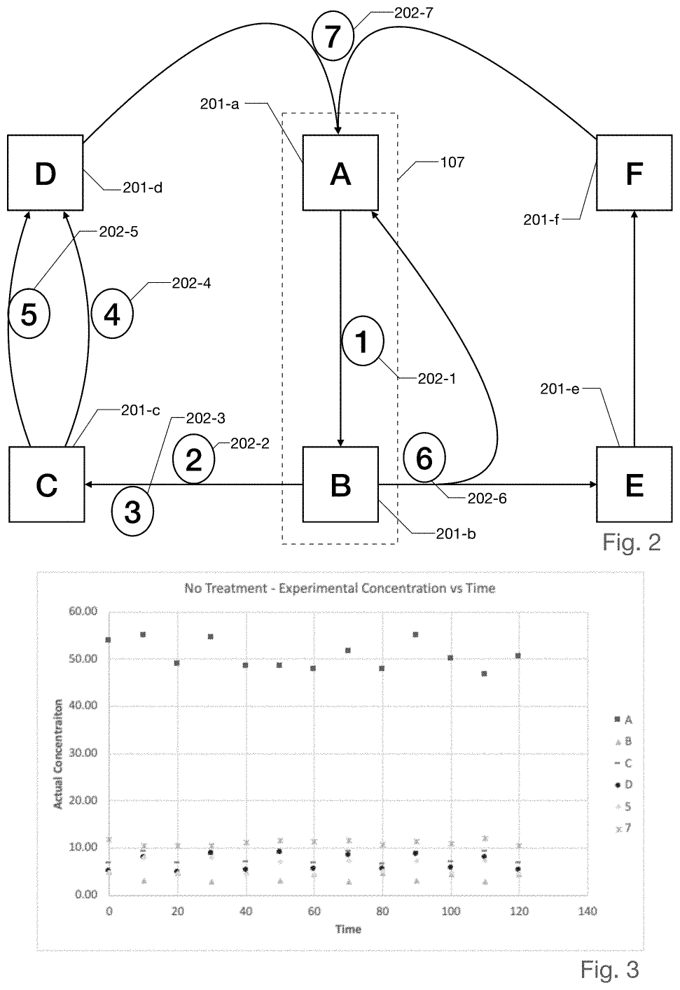

FIG. 2 illustrates an exemplary reaction model 200, in particular a pre-treatment reaction model 200a of biological network 102. For purposes of this disclosure, pre-treatment model 200a is a reaction model of the biological network 102 when biological model 102 is not undergoing treatment by drug therapy 101. As shown in FIG. 2, reaction model 200 represents a network of nodes 107, each a chemical interconversion of chemicals 201 in biological network 102. Such chemical interconversions are often facilitated by proteins 202. Protein 202 can be and often is an enzyme. Furthermore, chemical 201 can be a non-enzymatic protein 202. Reaction model 200 as shown, represents biological network 102 comprising seven nodes 107, as follows:
a. Node 1: Chemical A converts to chemical B, facilitated by protein 1.
b. Node 2,3: Chemical B converts to chemical C, facilitated by protein 2 and protein 3.
c. Node 4: Chemical C converts to chemical D, facilitated by protein 4
d. Node 5: Chemical C converts to chemical D, facilitated by protein 5
e. Node 6: Chemical B converts to chemical A and chemical E by protein 6.
f. Node 7: Chemical D and F together convert to chemical A, facilitated by protein 7.
g. Node X_{E}: Chemical E converts to chemical F without a modeled protein.

A person of ordinary skill in the art will recognize that not all chemical interconversions occurring within biological network 102 need be represented in reaction model 200. Furthermore, within each chemical interconversion represented within reaction model 200, not all chemicals 201 or proteins 202 involved in the chemical interconversion need be modeled within reaction model 200. For example, in process 1, A+Z₁→B may in fact be A+x₁+Z₁→B+y₁ wherein x₁ is a set of non-modeled reactants for processes 1, and y₁ is a set of non-modeled products for processes 1.

FIG. 3 illustrates a time-course progression 300 of biological network 102, specifically an actual time-course progression 300z. For purpose of this disclosure, actual time-course progression 300z is a time-course progression of concentrations of chemicals 201 experimentally-measured.

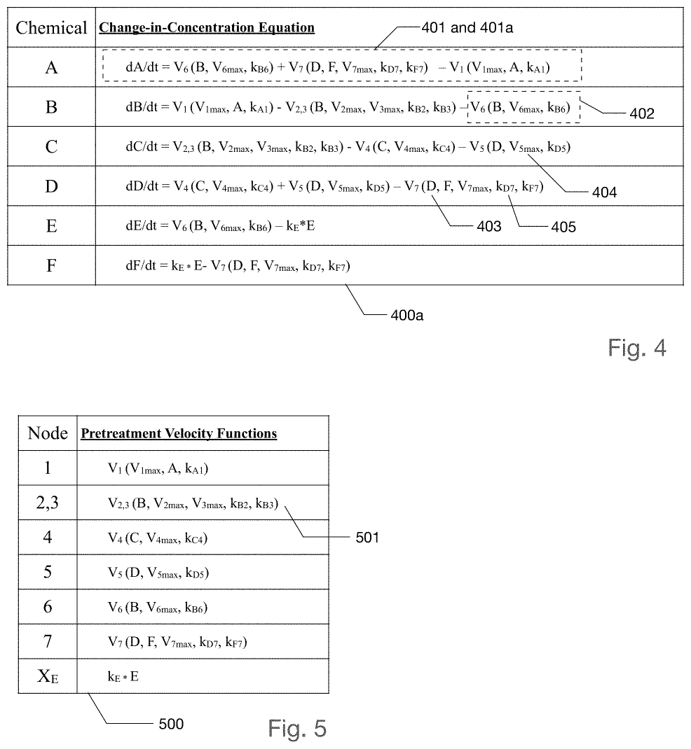

FIG. 4 illustrates a mathematical model 400 of biological network 102, in particular a pre-treatment mathematical model 400a. For purposes of this disclosure, mathematical model 400 relates to reaction model 200 and comprises equations 401 that together describe the behavior of biological network 102 with sufficient accuracy so as to be able to accurately predict the behavior of biological network 102 to which reaction model 200 relates. Further, for purposes of this disclosure, pre-treatment mathematical model 400a relates to pre-treatment reaction model 200a and comprises equations 401 that together describe the behavior of biological network 102 when not undergoing treatment by any drug therapy 101. Equations 401, in one embodiment, can comprise change-in-concentration equations 401a that each describe the rate of change in concentrations of a particular chemical 201 within reaction model 200. For example, change-in-concentration equations 301a relating to chemical A is as follows: dA/dt=V6(B, V6max, kB6)+V7(D, F, V7max, kD7, kF7)−V1(V1max, A, kA1).

Change-in-concentration equations 401a can comprise node-velocity functions Vₙ( ) 402 that describe the behavior of nodes 107 and are denoted in FIG. 4 as functions of various variables. For example, variables A through F each represent a concentration 403 of a corresponding chemicals 201 A-F in reaction model 201. V_{n max} represents a maximum node velocity 404 at which protein 202 can convert reactant(s) to product(s). Node velocity functions 402 each can be used to determine node velocity, and node velocities can be used to calculate changes in concentration of chemicals 201. One of ordinary skill in the art will recognize that node velocities can be modeled with a Michaelis-Menten equation. As an example, node velocity V₁ can be modeled with the equation V₁=(V₁ₘₐₓ*A)/(k_{A1}+A). In such equation, k_{A1} is a rate constant 405 specific to chemical A and protein 1. One of ordinary skill in the art will recognize that Both V₁ₘₐₓ and k_{A1} can be determined experimentally. These values can also be estimated. Furthermore, proteins 202 can operate on multiple chemicals or output multiple chemicals, leading to more complicated equations. Similarly, some nodes may require multiple proteins which can also lead to more complicated equations.

FIG. 5 illustrates a set of pretreatment node-velocity functions 500 for a pre-treatment mathematical model 400a model of biological network 102 not undergoing treatment, each node-velocity function is a pre-treatment velocity functions 501. For purposes of this disclosure, pre-treatment velocity function 501 is a node velocity function that models node 107 when such node is not being intervened-upon by drug regimen 103.

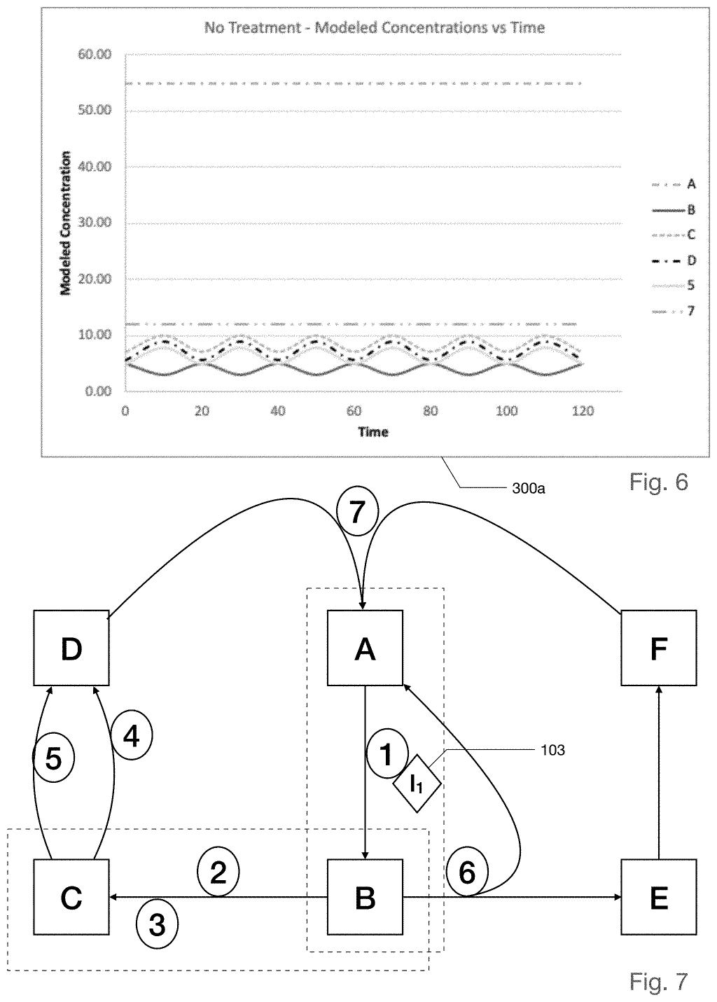

FIG. 6 illustrates a pre-treatment time-course progression 300a. One purpose of pre-treatment mathematical model 300a is to generate pre-treatment time-course progression 300a. For purpose of this disclosure, pre-treatment time course progression is a time course progression of pre-treatment mathematical model 300a and is intended to model with sufficient accuracy actual time-course progression 300z.

Time-course progressions 300 comprise levels of chemical concentrations of chemicals 201 within measured or modeled biological network 102 as a function of time. Pre-treatment time-course progression 300a models levels of chemical concentrations of chemicals 201 within biological network 102 as a function of time, prior to any treatment by drug therapy 101. Absent treatments, concentrations of chemicals 201 may vary in cycles, but otherwise typically remain within predictable, constrained levels for sustained periods within a life cycle of biological network 102. That is not to say that concentrations will remain constrained for an entire life cycle, but that during periods, concentrations will remain constrained, and that over changes from one period to the next remain substantially predictable. An important function of pretreatment time-course progression 400a is that it can establish baseline dynamics of biological network 102.

FIG. 7 illustrates an existing-treatment reaction model 200b. For purposes of this disclosure, existing treatment reaction model 200b is a reaction model that models how an existing drug therapy interacts with biological network 102. Existing-treatment reaction model 200b comprises at least one existing-treatment intervened-upon node 107a. Existing-treatment intervened-upon node 107a is intervened upon by drug regimen 103. Remaining nodes 107 that are not intervened upon are modeled as an untreated node 107b with a pre-treatment velocity function 501. It should be noted that existing drug therapy 101 is not exclusive only to drug therapies 101 that have reached the market but instead include any drug therapies previously considered for use on biological network 102 and lead to an outcome. In the case of target biological network 102a, examples of an outcome include but are not limited to causing target biological network 102a to die, rendering target biological network 102a unable to reproduce, or substantially undermine target biological network 102a such that other conditions or forces such as an immune system can kill target biological network 102a. In the case of a non-target biological network 102b, examples of an outcome can include strengthening non-target biological network 102b or rending non-target biological network 102b resistant or immune to some condition.

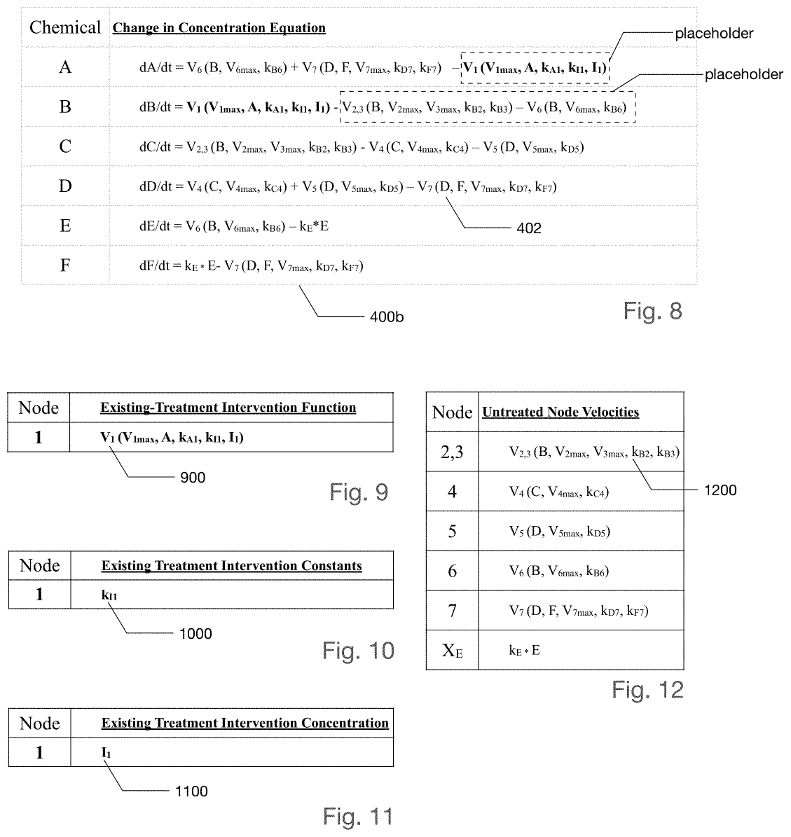

FIG. 8 illustrates an existing-treatment mathematical model 400b. In existing-treatment mathematical model 400b, each node-velocity function 402 modeling an existing-treatment intervened-upon node 107a can be an intervention function 402a. Types of intervention functions fall into two primary categories: an inhibition function or an acceleration function. Inhibition functions model the slowing or substantially stopping of a chemical interconversion at existing-treatment intervened-upon node 107a. Conversely, an acceleration function model the starting or speeding up of a chemical interconversion at existing treatment intervened-upon node 107a.

FIG. 9 illustrates a first set of existing-treatment intervention functions 900. As shown in FIG. 9, the first set 900 can be a set of one or more existing-treatment intervention functions.

FIG. 10 illustrates a set of existing-treatment intervention constants 1000. Each intervention function 402a can have one or more intervention constants 1001 related to one drug regimen 103 of existing drug therapy 101 related to existing-treatment mathematical model 400b. An intervention constant is 1001 a number that represents the effect on the speed of a node that related drug regimen 103 has on node 107.

FIG. 11 illustrates a set of existing-treatment intervention concentrations 1100. Each intervention function 402a can comprise a concentration constant 1102 that models a concentration of one drug regimen 103 of existing drug therapy 101. The set of existing-treatment intervention concentrations 1100 comprises these concentration constants 1102.

FIG. 12 illustrates a first set of untreated node velocity functions 1200. Within existing-treatment mathematical model 400b, untreated nodes 107b are modeled with pretreatment velocity functions 501. The first set of untreated node velocity functions 1200 comprises each of these pretreatment velocity functions 501.

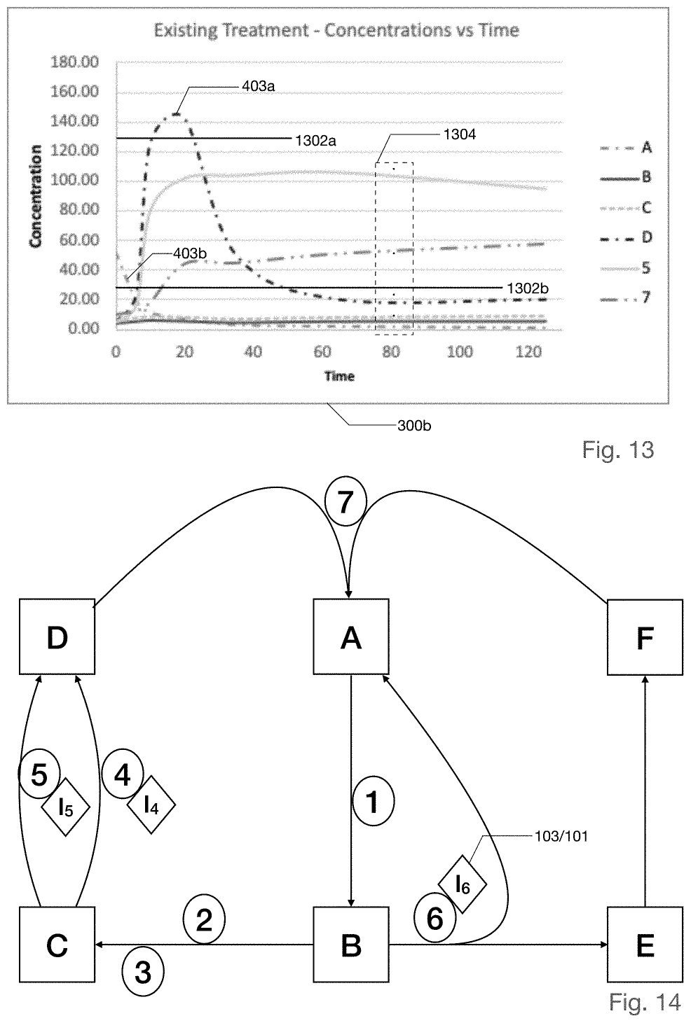

FIG. 13 illustrates an existing-treatment time-course progression 300b. Existing time-course progression 300b can comprise a time-course progression signature 1301 that predict outcomes as described above. Time-course progression signature 1301 can comprise one or more attributes. In one embodiment, time-course progression signature 1301 can be a first chemical concentration 403a of a first chemical 201 reaches a threshold 1302. In another embodiment, time-course progression signature 1301 can comprises a sequence of events. For example, the sequence of events can be defined at least in part by a first chemical 201a having a first chemical concentration 403a that meets or passes a first threshold 1302a followed by a second chemical 201b having a second chemical concentration 403b that meets or passes a second threshold 1302b. As another example, the sequence of events the defined at least in part by first chemical 201a having first chemical concentration 403a that meets or passes first threshold followed by the first chemical concentration a that meets or passes second threshold 1302b. In another embodiment, the time-course progression signature 1301 can comprise first chemical concentration 403a of first chemical 201a meeting or passing first threshold while second chemical concentration 403b of second chemical 201 is meeting or exceeding second threshold 1302b. In another embodiment, the time-course progression signature can comprise chemical concentrations 403 of chemicals 201 of a set of chemicals 1303 entering a range such that the chemical concentrations together do not deviate from a set of target concentrations 1304 for each of chemical 201 by equal to or more than a deviation threshold. In such embodiment, the deviation between the set of chemical concentrations 1303 and the set of target concentrations 1304 can be determined using a root-mean squared calculation.

In one embodiment, a set of parameters can be found for a new drug therapy such that the new drug therapy produces an outcome similar to an existing drug therapy. In a first step, the method can comprise replacing, within a mathematical model any intervention functions related to an existing drug therapy with untreated-node functions related to the mathematical model, if the mathematical model has any such intervention functions. In a next step, the method can comprise choosing parameter ranges for each of a plurality of parameters. Next, the method can comprise, producing a time course progression for a new-treatment mathematical model for each permutation of a plurality of permutations, each time, using the permutation to produce the time course progression. Next, the method can comprise determining for each permutation whether its time-course progression comprises a time-course progression signature present in an existing-treatment time-course progression related to the existing drug therapy, the time-course progression signature related to an outcome of the existing drug therapy. Next, the method can comprise, for at least one permutation comprising the time-course progression signature, synthesizing substances having kinetic properties substantially matching kinetic parameters of that one permutation, to produce a new drug therapy.

In one embodiment, parameter range can comprise a designation of a set of nodes that can be intervened upon. In another embodiment, parameter range can comprise ways to intervene, such as by speeding up or slowing down a chemical intervention within a node. In such embodiment, parameter range can comprise a plurality of intervention equations to be considered. In another embodiment, parameter range can comprise a range for one or more intervention constants for one or more intervention equations. In another embodiment, parameter range can include ranges for acceptable intervention concentrations. In another embodiment, parameter range can include spatial considerations.

FIG. 14 illustrates a new-treatment reaction model 200c. For purposes of this disclosure, new-treatment reaction model 200c is a reaction model that models how a new drug therapy might interact with biological network 102. New-treatment reaction model 200c comprises at least one new-treatment intervened-upon node 107a. New-treatment intervened-upon node 107a is intervened upon by drug regimen 103 of a new drug therapy 101. Remaining nodes 107 that are not intervened upon are modeled as an untreated node 107b with a pre-treatment velocity function 501.

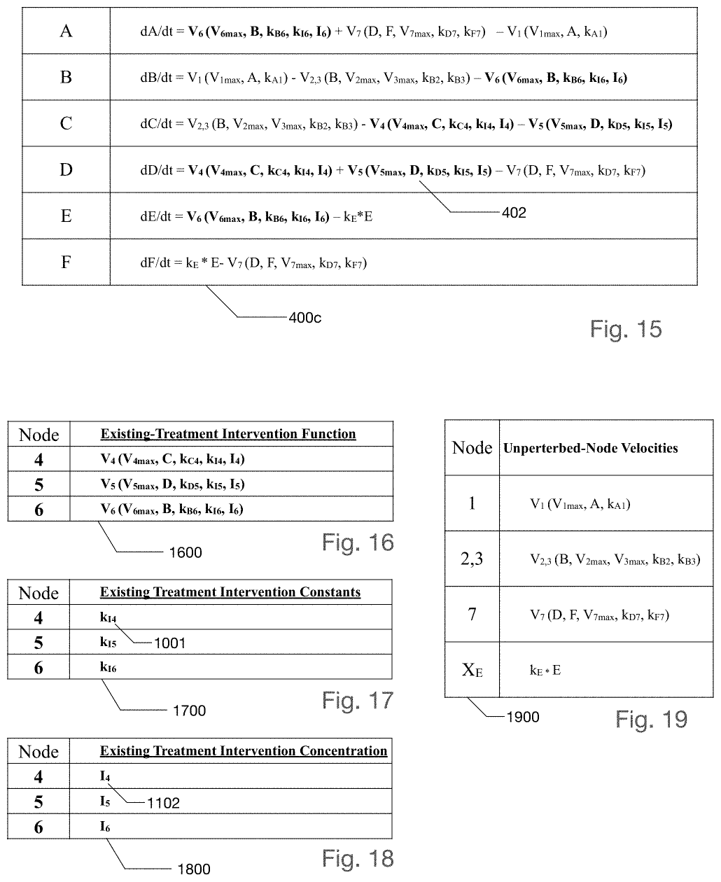

FIG. 15 illustrates new-treatment mathematical model 400c. In new-treatment mathematical model 400c, each node-velocity function 402 modeling new treatment intervened-upon node 107a can be an intervention function 402a. Types of intervention functions fall into two primary categories: an inhibition function or an acceleration function. Inhibition functions model the slowing or substantially stopping of a chemical interconversion at new-treatment intervened-upon node 107a. Conversely, an acceleration function model the starting or speeding up of a chemical interconversion at new-treatment intervened-upon node 107a.

FIG. 16 illustrates a second set of new-treatment intervention functions 1600. As shown in FIG. 16, the second set 1600 can be a set of one or more new-treatment intervention functions.

FIG. 17 illustrates a set of new-treatment intervention constants 1700. Each intervention function 402a can have one or more intervention constants 1001 related to one drug regimen 103 of new drug therapy 101 related to new-treatment mathematical model 400c. Intervention constant is 1001 a number that represents the effect on the speed of a node that related drug regimen 103 has on node 107.

FIG. 18 illustrates a set of new-treatment intervention concentrations 1800. Each intervention function 402a can comprise concentration constant 1102 that models a concentration of one drug regimen 103 of new drug therapy 101. The set of existing-treatment intervention concentrations 1800 comprises these concentration constants 1102.

FIG. 19 illustrates a second set of untreated node velocity functions 1900. Within new-treatment mathematical model 400b, untreated nodes 107b are modeled with pretreatment velocity functions 501. The first set of untreated node velocity functions 1200 comprises each of these pretreatment velocity functions 501.

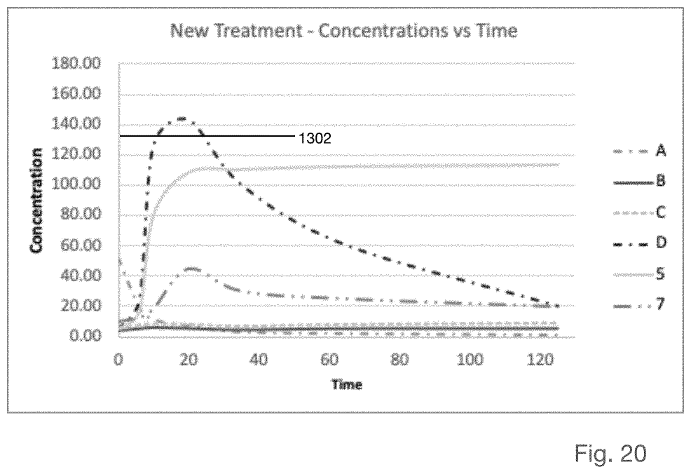

FIG. 20 illustrates a new-treatment time-course progression 300c. New time-course progression 300c can comprise time-course progression signature 1301 that predicts and is common, related to, or otherwise present in existing-treatment time-course progression 300b.

A method for developing a new drug therapy using characteristics of an existing drug therapy, the method comprising the following steps: developing a new-treatment mathematical model of a targeted biological network and synthesizing a drug therapy based on the new-treatment mathematical model. The new treatment mathematical model can be capable of producing a new-treatment time-course progression comprising a time-course progression signature found in an existing-treatment time-course progression of an existing treatment mathematical model of the targeted biological network. The time-course progression signature can be related to an outcome of the targeted biological network. The new-treatment mathematical model can comprise a new-treatment intervention function modeling each new-treatment intervened-upon node of a set of new-treatment intervened-upon nodes, and a first set of no-treatment node-velocity functions modeling all other nodes of the new-treatment mathematical model. Each new-treatment intervention function can comprise one more new-treatment intervention constants from a set of new-treatment intervention constants. The existing-treatment mathematical model can comprise an existing-treatment intervention equation modeling each existing-treatment intervened-upon node of a set of existing-treatment intervened-upon nodes, and a no-treatment velocity equation modeling all other nodes of the existing-treatment mathematical model. Each existing-treatment intervention equation can comprise an existing-treatment intervention constant from a set of existing-treatment intervention constants. The set of new-treatment nodes can be not identical to the set of existing-treatment nodes. Further, the set of new-treatment nodes can comprise at least one node not in the set of existing-treatment nodes. Further, the set of existing-treatment nodes can comprise at least one node not in the set of new-treatment nodes. A drug regimen can be synthesized for each the new treatment intervention constant, and the drug regimens together can be a new drug therapy.

This disclosure teaches new drug therapies designed using any of the afore-mentioned methods. For example, this disclosure teaches a drug therapy having a plurality of drug regimen, each regimen a substance having parameters ascertained using methods described above. Furthermore, the parameters are such that the new drug therapy has an outcome common to an existing drug therapy. Further, the drug regimen each intervene with a node, the nodes together a set of nodes, and such set of nodes are not in common with a second set of nodes intervened upon by the existing drug therapy.

A new drug development and design application, that can be stored in a memory, can provide properties and characteristics of theoretical therapeutic agent compounds that can be used to identify corresponding real-world potential therapeutic agent compounds. A high-resolution model developed from an identified biochemical or biological network interacting with a known therapeutic agent compound is produced and processed using the drug development and design system. Such method is referred to in this disclosure sometimes as a DASS method. The DASS method can provide a model and information about the effects of the known therapeutic with the identified biochemical or biological network. In one embodiment, the DASS method can also be used in providing models and information about identified biochemical or biological networks without known therapeutics. After the processing using the DASS method, characteristics and properties of theoretical therapeutic agent compounds which affect the identified biochemical or biological network the same way the original therapeutic affects the networks can be identified. Similarly, theoretical therapeutic agents for the particular target cells in the identified biochemical or biological networks without known therapeutics can be identified using the method.

In an embodiment of the DASS method, the system outputs the theoretical properties and characteristics of theoretical therapeutic agent compounds that can be used to interact with the identified biochemical or biological networks. The said properties and characteristics of the theoretical therapeutic agent compounds can be used to compare with real world therapeutic agent compounds and consequently identify potential therapeutic agent compounds, that produces a similar pathway behavior to the biological or biochemical network or at least the target cell, which can be tested on the identified biochemical or biological networks.

In another embodiment, the potential therapeutic agent compounds are simulated against the identified biochemical or biological networks to check if the potential therapeutic agent compounds can be a high-quality drug candidate. A high-quality drug candidate is the identified potential therapeutic agent compound that can produce the same or better effects like the original therapeutic agent compound against a target cell, and causes minimal perturbation on non-target cells, or it produces an overall similar pathway behavior on the identified biochemical or biological network as the original therapeutic does.

In another embodiment, the modelling and simulation of the biochemical or biological networks are preformed by the drug development and design system, and all information regarding any modelling performed can be stored in the data storage of the system.

A system to perform the method described herein could comprise a typical networking environment comprising a number of electronic devices and a server connected over a network. Examples of electronic devices can include, but are not limited to, a computer, a smart phone, and/or a tablet. In one embodiment, electronic device, and server can communicate with each other. Network 107 can be hardwired, wireless, or a combination of both. An example of a LAN is a network within a single building. An example of a WAN is the Internet.

An electronic device can comprise a local memory and a local processor. The local memory can comprise a local application and a local data.

A server can comprise a server memory and a server processor. Server memory can comprise a server application and a server data.

In one embodiment, a drug development and design application can mean local application wherein interface, presentation, logic, and data storage are controlled locally on the electronic device. In such embodiment, memory can mean local memory, processor can mean local processor, and data can mean local data.

In another embodiment, the drug development and design application can mean local application together with server application. One example of such embodiment is where local application is a general-purpose (browser) application. Another example of such embodiment is where local application is a specific-purpose (non-browser) application.

In the first example, a browser accesses the server application via a website. In such embodiment, interfacing with a user would occur using the electronic device, presentation can be performed by local application and server application, while the logic and data can be performed by server. In such example, memory could mean electronic device memory and/or server memory, processor could mean electronic device processor and/ore server processor, and data could mean server data.

In the second example, the specific-purpose application accesses server application 103b. In such embodiment, interfacing with the user can occur on electronic device, while presentation, logic, and data storage can be distributed both on electronic device and server. In such example, memory would mean electronic device memory and/or server memory, processor could mean electronic device processor and/or server processor, and data would mean local data and/or server data.

Stored in the memory described herein above are both data and several components that are executable by the processor. In particular, stored in the memory and executable by the processor is the DASS method and potentially other applications. Also stored in the memory can be information such as interaction of known therapeutic agent compounds with target cells, kinetic characterization data of therapeutic agent compounds with non-target cells, and other data. In addition, an operating system can be stored in the memory and executable by the processor.

Although the drug development and design system and other various systems described herein can be embodied in software or code executed by general purpose hardware as discussed above, as an alternative the same can also be embodied in dedicated hardware or a combination of software/general purpose hardware and dedicated hardware. If embodied in dedicated hardware, each can be implemented as a circuit or state machine that employs any one of or a combination of a number of technologies. These technologies can include, but are not limited to, discrete logic circuits having logic gates for implementing various logic functions upon an application of one or more data signals, application specific integrated circuits having appropriate logic gates, or other components, etc. Such technologies are generally well known by those skilled in the art and, consequently, are not described in detail herein.

Also, any logic or application described herein, including the drug development and design system, that comprises software or code can be embodied in any computer-readable storage medium for use by or in connection with an instruction execution system such as, for example, processor in a computer system or other system. In this sense, the logic can comprise, for example, statements including instructions and declarations that can be fetched from the computer-readable storage medium and executed by the instruction execution system.

It should be emphasized that the above-described embodiments of the present disclosure are merely possible examples of implementations set forth for a clear understanding of the principles of the disclosure. Many variations and modifications can be made to the above-described embodiment(s) without departing substantially from the spirit and principles of the disclosure. All such modifications and variations are intended to be included herein within the scope of this disclosure and protected by the following claims.

Various changes in the details of the illustrated operational methods are possible without departing from the scope of the following claims. Some embodiments may combine the activities described herein as being separate steps. Similarly, one or more of the described steps may be omitted, depending upon the specific operational environment the method is being implemented in. It is to be understood that the above description is intended to be illustrative, and not restrictive. For example, the above-described embodiments may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein”.

1. A method for developing a new drug therapy using characteristics of an existing drug therapy, the method comprising the following steps:
developing a new-treatment mathematical model of a targeted biological network, said new treatment mathematical model capable of producing a new-treatment time-course progression comprising a time-course progression signature found in an existing-treatment time-course progression of an existing treatment mathematical model of said targeted biological network, said time-course progression signature related to an outcome of said targeted biological network;
said new-treatment mathematical model comprising a new-treatment intervention function modeling each new-treatment intervened-upon node of a set of new-treatment intervened-upon nodes, and a first set of no-treatment node-velocity functions modeling all other nodes of said new-treatment mathematical model, each said new-treatment intervention function comprising one or new-treatment intervention constants from a set of new-treatment intervention constants;
said existing-treatment mathematical model comprising an existing-treatment intervention equation modeling each existing-treatment intervened-upon node of a set of existing-treatment intervened-upon nodes, and a no-treatment velocity equation modeling all other nodes of said existing-treatment mathematical model, each said existing-treatment intervention equation comprising an existing-treatment intervention constant from a set of existing-treatment intervention constants;
said set of new-treatment nodes not identical to said set of existing-treatment nodes, further said set of new-treatment nodes comprising at least one node not in said set of existing-treatment nodes, further said set of existing-treatment nodes comprising at least one node not in said set of new-treatment nodes; and
synthesizing a drug regimen for each said new treatment intervention constant, said drug regimens together a new drug therapy.

2. The method of claim 1 wherein
said set of new-treatment time-course progressions comprises a new-treatment time-course progression for each chemical of a set of chemicals within said targeted biological network; and
said set of existing-treatment time-course progressions comprises an existing-treatment time-course progression for each said chemical of said set of chemicals.

3. The method of claim 2 wherein
said set of new-treatment time-course progressions further comprises a new-treatment time-course progression for each protein of a set of proteins within said targeted biological network; and
said set of existing-treatment time-course progressions further comprises an existing-treatment time-course progression for each said protein of said set of proteins.

4. The method of claim 2 wherein said time-course progression signature comprises a first chemical concentration of a first chemical of said chemicals reaching or passing a threshold.

5. The method of claim 2 wherein said time-course progression signature comprises a sequence of events.

6. The method of claim 5 wherein said sequence of events is defined at least in part by a first chemical having a first chemical concentration that meets or passes a first threshold followed by a second chemical having a second chemical concentration that meets or passes a second threshold.

7. The method of claim 5 wherein the sequence of events is defined at least in part by a first chemical having a first chemical concentration that meets or passes a first threshold followed said first chemical concentration that meets or passes a second threshold.

8. The method of claim 2 wherein said time-course progression signature comprises a first chemical concentration of a first chemical of said set of chemicals meeting or passing a first threshold while a second chemical concentration of a second chemical of said chemicals is meeting or exceeding a second threshold.

9. The method of claim 2 wherein said time-course progression signature comprises chemical concentrations of said chemicals of said set of chemicals entering a range such that said chemical concentrations together do not deviate from a set of target concentrations for each of said chemicals by equal to or more than a threshold.

10. The method of claim 9 wherein a deviation between said chemical concentrations and said set of target concentrations is determined using a root-mean squared calculation.

11. The method of claim 1 wherein at least one of said new-treatment intervention equations is an inhibition equation and said intervention constant associated with said inhibition equation is an inhibition constant.

12. The method of claim 1 wherein at least one of said new-treatment intervention equations is an acceleration equation and said intervention constant associated with said acceleration equation is an acceleration constant.

13. The method of claim 1 wherein at least one of said no-treatment equations is a Michaelis-Menten equation.

14. The method of claim 1 wherein said set of existing-treatment nodes comprises only one node.

15. The method of claim 1 wherein there exists no nodes in common with said set of existing treatment nodes and said set of new-treatment nodes.

16. The method of claim 1 wherein each node in common between said set of existing treatment nodes and said set of new treatment nodes comprise a different existing-treatment intervention constant as compared to a corresponding new-treatment intervention constant.

17. A method for finding a set of parameters for a new drug therapy such that the new drug therapy produces an outcome similar to an existing drug therapy, comprising the steps:
replacing, within a mathematical model any intervention functions related to an existing drug therapy with untreated-node functions related to the mathematical model, if the mathematical model has any such intervention functions;
choosing parameter ranges for each of a plurality of parameters;
producing a time course progression for a new-treatment mathematical model for each permutation of a plurality of permutations, each time, using said permutation to produce said time course progression;
determining for each permutation whether its time-course progression comprises a time-course progression signature present in an existing-treatment time-course progression related to said existing drug therapy, said time-course progression signature related to an outcome of said existing drug therapy; and
synthesizing, for at least one permutation comprising said time-course progression signature, substances having kinetic properties substantially matching kinetic parameters of that one permutation, to produce a new drug therapy.

18. The method of claim 17 wherein said parameter ranges comprise a designation of a set of nodes that can be intervened upon.

19. The method of claim 17 wherein said parameter ranges comprise a plurality of intervention equations for consideration.

20. The method of claim 17 wherein parameter ranges comprise a range for one or more intervention constants for one or more intervention equations.

21. The method of claim 17 wherein parameter ranges comprise intervention constant ranges.

22. The method of claim 17 wherein parameter ranges comprise spatial considerations.
